(19) United States
(12) Patent Application Publication
(10) Pub. No.: US 2015/0366684 A1
(43) Pub. Date: Dec. 24, 2015

(54) STENT

(71) Applicant: SAMSUNG LIFE WELFARE FOUNDATION, Seoul (KR)

(72) Inventors: Soo Won SEO, Seongnam (KR); In Wook CHOO, Seoul (KR); Jae Jun KIM, Seoul (KR); Jin Yong KIM, Seoul (KR); Hong Suk PARK, Seoul (KR)

(21) Appl. No.: 14/841,770

(22) Filed: Sep. 1, 2015

Related U.S. Application Data
(63) Continuation-in-part of application No. 13/167,802, filed on Jun. 24, 2011, now abandoned.

(30) Foreign Application Priority Data
Jun. 25, 2010 (KR) 10-2010-0060742

(51) Int. Cl. A61F 2/89; A61F 2/00

(52) U.S. Cl. CPC A61F 2/89; A61F 2/0077; A61F 2210/0014; A61F 2230/0069; A61F 2250/001; A61F 2250/0039; A61F 2310/00389; A61F 2002/009

(57) ABSTRACT

A stent is disclosed herein. The stent includes a silicon cover, a plurality of cylindrical members, and connection members. The silicon cover is formed in an open hollow cylindrical shape in which the diameter of both ends thereof is larger than the diameter of the intermediate portion thereof. The plurality of cylindrical members is disposed on the outer circumference of the silicon cover in a hollow cylindrical shape whose both ends are open, and is configured such that the diameter thereof is reduced by external force to be installed in a tubule. The connection members connect facing ones of the plurality of cylindrical members at regular intervals in a single ring shape between the cylindrical members, and have elasticity in a lengthwise direction.

### CROSS-REFERENCES TO RELATED APPLICATION

This application is a Continuation-in-part application of U.S. patent application Ser. No. 13/167,802 filed on 2011 Jun. 24, which claims priority to Korean Patent Application No. 10-2010-0060742 filed on Jun. 25, 2010, the contents of which are incorporated herein by reference.

### BACKGROUND

1. Technical Field

The present invention relates to a stent and, more particularly, to a stent that is configured such that a plurality of cylindrical members disposed in a silicon cover is connected by ring-shaped connection members, thereby preventing a fatigue failure by ensuring predetermined durability while maintaining flexibility in a lengthwise direction, and also preventing re-stenosis or the growth of a tumor.

2. Description of the Related Art

Generally, there are cases where a tubule (a lumen, a blood vessel, or the like) in a human body is narrowed due to disease, injury, an operation, or the like. When a tubule is narrowed, the function thereof may be deteriorated or cannot be performed. In these cases, various devices for expanding a narrowed tubule and/or preventing an expanded tubule from being narrowed again are used. A stent is a representative of these devices.

When a tubule is narrowed, for example, when the narrowness of the esophagus occurs due to cancer of the esophagus, when blood does not circulate smoothly due to the hardening of an artery or when a tract through which bile from the liver passes is narrowed, a stent is installed in the narrowed tubule, so that the tubule is expanded and the expanded state is maintained, thereby enabling food, blood or bile to smoothly flow through the tubule.

Stents having different structures and characteristics may be used according to a method for installing a stent as well as the position and type of a tubule.

For example, when a stent is installed in an alimentary tract, a stent having flexibility in a lengthwise direction is used so as to be flexible in response to the movement of the alimentary tract. In the past, a stent was installed in a tubule using X-ray imaging medical equipment. In contrast, since a device for inserting an endoscope has recently become narrow, a stent may be installed through the side channel of an endoscope, in which case a stent may be used by considering the inner diameter of the device for inserting an endoscope.

As described above, different types of stents may be used depending on the position and type of a tubule and a method of installing a stent. There are various criteria for selecting a stent, such as flexibility in the lengthwise direction, expandability, thickness, durability, and so on.

Among these criteria, the flexibility in a lengthwise direction and the durability are the most sensitive criteria for a patient in which a stent is installed. The flexibility in a lengthwise direction is important in that a patient may feel a sense of irritation due to the insertion of a stent, and the durability is important in that it determines the time at which a stent needs to be replaced.

However, when the flexibility in the lengthwise direction increases, the dynamic fatigue of a stent may increase and the durability of a stent may be deteriorated. In contrast, when the durability increases, the strength of the material and structure of a stent may increase and the flexibility in a lengthwise direction may decrease. That is, the flexibility in a lengthwise direction and the durability have an inverse relationship.

Therefore, there is a demand for the development of a stent that meets the two sensitive criteria for a patient.

In the case of a stent used in an alimentary tract connected to a stomach in response to the above-described demand, a covered stent is used in the state in which the stent has been separated into a plurality of segments. For example, like a conventional stent shown in FIG. 5, a silicon cover 10 is inserted into a stent 20 separated into a plurality of segments, the stent 20 is covered with the silicon cover 10, and the segmented stent is connected by helical wire 30.

However, this type of stent is problematic in that, when the silicon cover 10 between the segments of the stent 20 is destructed, the individual segments of the stent are freely rotated and moved in a lengthwise direction, and thus the function of the stent cannot be appropriately performed.

Furthermore, placement of stent has been established as an important palliative treatment in benign and malignant obstruction of the GI tract. Although both covered and uncovered stents can be used, covered stents have two main advantages—easy retrievability, which is a prerequisite for benign lesions, and a lower rate of re-stenosis in malignant lesions than uncovered ones, because its membrane prevents the ingrowth of a tumor through the mesh. However, covered stents have a higher risk of migration compared with uncovered ones, and migration is one of the most common adverse events in covered stent placement in most studies.

The conventional stent, which is constructed with multiple segments of covered stent without metallic connections between each stent segment, has been assumed to have excellent anti-migration properties. However, the gap between individual stent segments made of only polymer membranes is both the key element of stent flexibility and anti-migration property, and is the weakest part of the stent where disruption may occur.

### SUMMARY

Accordingly, the present invention has been made keeping in mind the above problems occurring in the prior art, and an object of the present invention is to provide a stent that is capable of preventing a fatigue failure by ensuring predetermined durability while maintaining flexibility in a lengthwise direction, and is also capable of maintaining the shape of the stent without change even in any situation.

Furthermore, another object of the present invention is to reinforce the conventional stent with linking rings between stent segments without reducing its flexibility and anti-migration properties, and to compare the physical properties and migration rates of the stent of the present invention and the conventional stent in an animal colon obstruction model.

In accordance with an aspect of the present invention, there is provided a stent, including: a silicon cover formed in an open hollow cylindrical shape in which the diameter of both ends thereof is larger than the diameter of the intermediate portion thereof; a plurality of cylindrical members disposed on the outer circumference of the silicon cover in a hollow cylindrical shape whose both ends are open, and configured such that the diameter thereof is reduced by external force to be installed in a tubule; and connection members configured to connect facing ones of the plurality of cylindrical members at regular intervals in a single ring shape between the cylindrical members and to have elasticity in a lengthwise direction.

The cylindrical members may be each fabricated by disposing one or more lines along a circumferential surface in a zigzag shape to form a plurality of peak parts and a plurality of valley parts.

The connection members may connect the most adjacent peak and valley parts of the facing ones of the cylindrical members.

The connection members may include at least three connection members that are connected along the circumferences of the cylindrical members.

The connection members that connect the cylindrical members may be disposed at the same height in a single rectilinear row.

The connection members may be disposed in two or more rows.

The cylindrical members may be made of metallic or resin wire that is harmless to a human body.

The connection members may be made of metallic or resin wire that is harmless to a human body.

The metallic wire of the cylindrical members or connection members may be made of nitinol or stainless steel.

The cylindrical members may be configured such that, when external force is applied, the angles of the peak and valley parts are varied, thereby varying the diameter of the cylindrical members.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a plan view showing a stent according to a first embodiment of the present invention;

FIG. 2 is a cross sectional view showing the stent according to the first embodiment of the present invention;

FIG. 3 is a plan view showing a stent according to a second embodiment of the present invention;

FIG. 4 is a plan view showing a stent according to a third embodiment of the present invention;

FIG. 5 is a plan view showing a conventional stent;

FIG. 6 is a photo showing a fatigue test in which after stents were installed in a testing machine by fixing them with custom-made jigs, the compression and elongation of the stents at angles of 60° was repeated with a frequency of 10 Hz for 10 days; and

FIG. 7 shows photos of animal study, in which “A” is a fluoroscopic view showing the surgical obstruction and proximal dilatation of the colon, “B” indicates that after the placement of the novel stent of the present invention, obstruction disappeared, and “C” indicates that one week after stent placement, the stent was patent and in position.

### DETAILED DESCRIPTION

Embodiments of the present invention will be described in detail below with reference to the accompanying drawings. In the following description of the embodiments of the present invention, descriptions of well-known functions and/or configurations may be omitted in order to make the gist of the present invention clear.

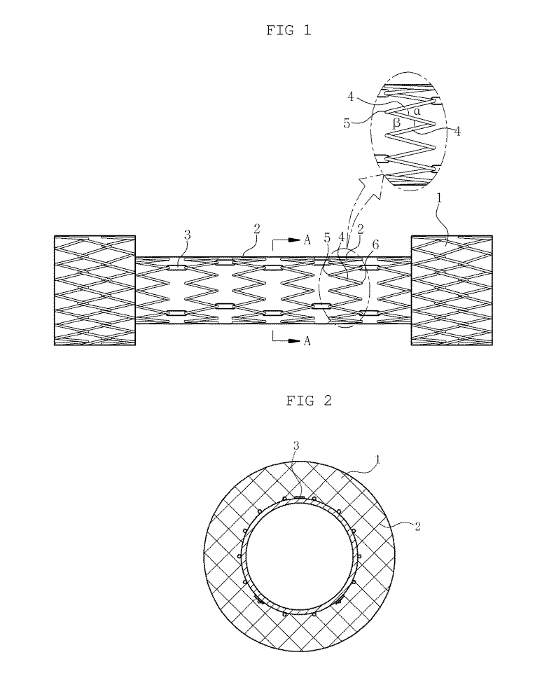

First, a stent according to an embodiment of the present invention is described in detail with reference to FIGS. 1 and 2.

In this case, FIGS. 1 and 2 are a plan view and a cross sectional view showing the stent according to the present embodiment.

As illustrated in FIGS. 1 and 2, the stent according to the present embodiment includes a silicon cover 1, a plurality of cylindrical members 2, and at least three connection members 3 each configured to connect adjacent ones of the plurality of cylindrical members 2 to each other.

The silicon cover 1 is formed of a silicon membrane material appropriate for a living body in a hollow, cylindrical shape whose both ends are open, and is configured such that the both ends thereof are formed to have a diameter larger than that of the intermediate portion, thereby being suitable for a stent that will be applied to the alimentary tract.

The silicon cover 1 is inserted into and covered to the plurality of cylindrical members 2, and the plurality of cylindrical members 2 is disposed in a rectilinear or non-rectilinear row. The plurality of the cylindrical members 2 forms the appearance of the stent according to the present embodiment along with the silicon cover 1, and is hollow-shaped members. The plurality of cylindrical members 2 that form the stent segmented into a plurality of elements comes into tight contact with the outer circumference of the silicon cover 1 in a state in which the intermediate portion of the stent is separated into a plurality of segments between both ends at regular intervals. Accordingly, the cylindrical members 2 are also configured such that the side cylindrical members 2 have a diameter larger than that of the intermediate cylindrical members 2.

The circumferential surface of the cylindrical member 2 is a surface that comes into contact with the side surface of a tubule. When external force equal to or higher than a preset magnitude is applied to the cylindrical members 2, a space inside the cylindrical member 2 can be reduced, i.e., the diameter of the circumferential surfaces of the cylindrical members 2 can be reduced.

In this case, the “external force” refers to force that is applied to the cylindrical members 2 in order to easily insert the stent according to an embodiment of the present invention into a tubule. When external force equal to or higher than a preset magnitude is applied to the cylindrical members 2, the diameter of the circumferential surface of the cylindrical members 2 is reduced, and thus the stent may be easily inserted into a tubule. Furthermore, the “preset magnitude of external force” refers to the magnitude of force that can sufficiently reduce the diameter of the circumferential surfaces of the cylindrical members 2 when an apparatus for inserting the stent applies force to the cylindrical members 2. The preset magnitude of external force is set depending on the structure and material of the cylindrical members 2.

The connection members 3 includes at least three connection members that are disposed at regular intervals along the circumference of the cylindrical member 2 so that oval ring-shape wire connects facing ones of the plurality of cylindrical members at regular intervals between both ends of the cylindrical members 2. The connection members 3 are disposed in a single rectilinear row because they connect the plurality of cylindrical members 2 at the same height between the plurality of cylindrical members 2. Furthermore, the ring-shaped wire is formed to have a single closed curve shape by connecting both ends of wire to each other.

Referring to FIGS. 1 and 2, the cylindrical members 2 and the connection members 3 are described in detail below.

As illustrated in FIGS. 1 and 2, each of the cylindrical members 2 includes one or more lines 4. Each of the cylindrical members 2 is formed by forming a closed curve in such a way as to connect both ends of the line 4 to each other and disposing the closed curve to have a predetermined length in the lengthwise direction of the cylindrical member 2. The closed curve is disposed on the circumferential surface of the cylindrical member 2. The line 4 has a preset thickness. The preset thickness is set by those skilled in the art by taking into account desired rigidity, the material of the line, the disposition of the closed curve, etc.

As described above, the cylindrical members 2 may be each fabricated by disposing a closed curve, composed of a single line 4, on a circumferential surface. The closed curve may be disposed in a zigzag shape having a plurality of peak parts 5 and a plurality of valley parts 6. In this case, the peak parts 5 refer to parts convex in the lengthwise direction of the cylindrical member 2 (i.e., parts convex to the left of the diagram), and the valley parts 6 are parts concave in the lengthwise direction of the cylindrical member 2 (i.e., parts convex to the right of the diagram). The angles α and β of the peak parts 5 and the valley parts 6 may be varied when external force is applied. That is, when external force is applied to the cylindrical member 2, the angles α and β of the peak parts 5 and the valley parts 6 are reduced and the diameter of the circumferential surface of the cylindrical member 2 is reduced. Furthermore, when the angles α and β of the peak parts 5 and the valley parts 6 are increased by external force, the diameter of the circumferential surface of the cylindrical member 2 is also increased. When external force applied to the cylindrical member 2 is removed, the cylindrical member 2 is returned to its original shape. That is, the cylindrical member 2 has elasticity.

As illustrated in FIGS. 1 and 2, the connection members 3 allow the stent to have a single pipe shape by connecting adjacent cylindrical members 2 to each other. The cylindrical members 2 are allowed to be relatively moved by the connection members 3. Accordingly, the connection members 3 allow the shape of the stent to be adjusted in conformity with the shape of a tubule.

When ring-shaped wires are used as the connection members 3, durability can be improved. The connection members 3 may connect the most adjacent peak parts 5 and valley parts 6 of facing cylindrical members 2.

The connection members 3 are made of a metallic material or resin material by taking into account durability. It is preferred that such a metallic material or resin material be harmless to a human body.

The connection members 3 may be made of a material having elasticity so that they may be sufficiently extended in the lengthwise direction of the cylindrical member 2. When ring-shaped wires are used as the connection members 3, the connection members 3 may be extended in the lengthwise direction of the cylindrical members 2 to some extent due to characteristics in terms of shape. When the connection members 3 are extended or contracted in the lengthwise direction of the cylindrical members 2, the stepwise installation of the stent may be simplified. That is, after a single cylindrical member 2 has been installed at a desired location, the installation location of another single cylindrical member 2 may be simply adjusted.

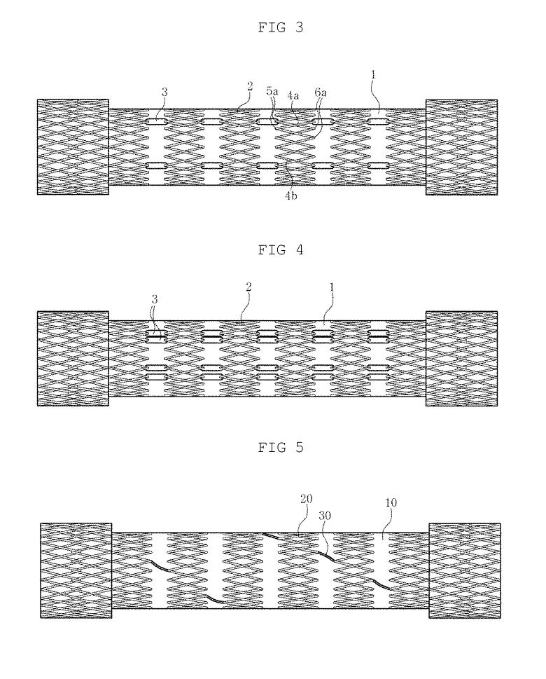

As illustrated in FIG. 3, cylindrical members 2 may be each fabricated by disposing closed curves composed of two lines 4a and 4b on a circumferential surface. In this case, the closed curves may be disposed in the same shape or in different shapes. Furthermore, the closed curves may be disposed in a circumferential direction at one or more regular intervals. In this case, the closed curves are connected and come into contact with each other at intersection points. The closed curves may be each disposed in a zigzag shape having a plurality of peak parts 5a and a plurality of valley parts 6a. As illustrated in FIG. 2, the peak parts 5a and the valley part 6a may have sharp tips. Furthermore, as illustrated in FIG. 3, the peak parts 5a and the valley parts 6a may have blunt tips. The angles α and β of the peak parts 5a and the valley parts 6a may be varied when external force is applied. The diameter of the circumferential surfaces of the cylindrical members 2 is reduced or increased by variations in the angles α and β of the peak parts 5a and the valley parts 6a.

The size of the cylindrical members 2 may be varied depending on the location and type of a target tubule. The length and diameter of the circumferential surfaces of the cylindrical members 2 may be adjusted by the disposition of closed curves. The disposition of closed curves is not limited to the disposition that is illustrated as an example herein. The disposition of closed curves that allows the diameter of the circumferential surfaces of the cylindrical members 2 may be varied by external force should be understood as falling within the scope of the present invention.

Meanwhile, the cylindrical members 2 need to be open at their both ends so that food, blood or bile can pass through the cylindrical members 2.

Furthermore, the material of the cylindrical members 2 may be varied depending on the location and type of a target tubule. In some embodiments, the cylindrical members 2 may be made of a metallic material or resin material in order to ensure durability and rigidity. In this case, it is preferred that the metallic material or resin material be harmless to a human body.

The one or more cylindrical members 2 are disposed to form a single row, as described above. Compared to the use of a single cylindrical member 2, the use of a plurality of cylindrical members 2 facilitates the adjustment of the location of the stent in a tubule.

That is, when a single cylindrical member 2 is used, a stent is installed in a tubule once, so that careful attention should be paid so that the stent can be installed at a desired location during first installation. In contrast, when two or more cylindrical members 2 are used, the cylindrical members 2 are sequentially installed in a tubule, so that it is easy to adjust the location of each of the cylindrical members 2.

When the cylindrical members 2 and the connection members 3 are made of metallic wire, it is preferable to use nitinol or stainless steel.

FIG. 4 is a plan view showing a stent according to a third embodiment of the present invention.

As illustrated in FIG. 4, in the stent according to the third embodiment of the present invention, connection members that consecutively connect a plurality of adjacent cylindrical members 2 to each other are disposed in two or more rows, other than in a single row. The number of rows in which the connection members 3 are disposed may be set by a designer depending on the type and location of a tubule in which a stent will be installed. That is, when flexibility in the lengthwise direction is more necessary than durability, the connection members 3 may be disposed in a single row. In contrast, in the case of a tubule in which durability is more necessary than flexibility in the lengthwise direction, the connection members 3 may be disposed in two or more rows.

The process of inserting a stent according to an embodiment of the present invention into a tubule and the operation of the stent will be described in detail below with reference to FIGS. 1 to 4.

To insert a stent according to an embodiment of the present invention into a tubule, the diameter of the cylindrical members 2 is reduced by applying external force to the cylindrical members 2, together with the silicon cover 1 in a radially inward direction. As described above, when external force is applied to the cylindrical members 2 in a radially inward direction, the angles α and β of peak parts 5 and valley parts 6 are reduced, and thus the diameter of the circumferential surfaces of the cylindrical members 2 is reduced.

Thereafter, the locations of installation of the cylindrical members 2, together with a silicon cover 1, are determined. Thereafter, the cylindrical members 2 whose diameter has been reduced are inserted into a tubule in conformity with the locations of installation, and the connection members 3 are located at target locations. In this case, with respect to the locations of installation, a lesion, such as a tumor, is caught within an intermediate portion having a smaller diameter and the lesion is interposed between both ends having a larger diameter, so that the lesion, such as the tumor, is caught by stepped portions between the ends having a larger diameter and the intermediate portion having a smaller diameter and thus the stent is prevented from being moved.

The external force applied to the cylindrical members 2 is removed in order from the cylindrical member 2 inserted to the highest depth to the cylindrical member 2 inserted into the lowest depth. In this case, the angles α and β of the peak parts 5 and valley parts 6 of the cylindrical members 2 from which external force has been removed is increased again, and the individual cylindrical members 2 are fitted into a tubule. That is, the stent is completely fitted into the tubule. In some embodiments, external force applied to the cylindrical members 2 is removed in order from the cylindrical member 2 inserted into the highest depth to the cylindrical member 2 inserted into the lowest depth. Once the stent has been installed in a tubule, the tubule is expanded and is not further narrowed. In particular, the re-stenosis of a lesion, such as a tumor, into the cylindrical members 2 or the growth thereof is prevented by the silicon cover 1.

As described above, the plurality of cylindrical members 2 connected by the connection members 3 may be relatively moved to some extent. Accordingly, even when the movement of a tubule in which a stent according to an embodiment of the present invention has been installed is increased, the possibility that the stent is separated from the tubule is reduced. That is, the individual cylindrical members 2 may be freely moved in response to the movement of the tubule.

Meanwhile, the plurality of cylindrical members 2 that can be freely moved to some extent in the above stent is connected by the connection members 3, and the connection members 3 are extended or contracted by the movement of the cylindrical members 2 attributable to the movement of the tubule. Accordingly, the connection members 3 may be easily subjected to serious dynamic fatigue.

As described above, in some embodiments, the connection members 3 are formed in a ring shape, thereby preventing stress concentration and also reducing dynamic fatigue.

For example, when rectilinear connection members are used, stress concentration may occur in the connection members due to the movement of the cylindrical member 2 attributable to the movement of a tubule. Furthermore, when stress concentration occurs repeatedly, the connection members 3 may be destructed by fatigue. In order to prevent the fatigue failure of the connection members 3, the ring-shaped connection members 3 are employed.

According to an embodiment of the present invention, a plurality of cylindrical members is connected by at least three connection members, flexibility in a lengthwise direction can be maintained and durability can be ensured.

Next, the physical properties and migration rates of the stent of the present invention installed in the alimentary tract using the above-described method, the conventional stent of FIG. 5 configured such that segmented cylindrical members are connected by helical wire, and a Bonastent composed of a single cylindrical member without a connection member were tested and compared as follows.

That is, the present inventors tested the physical properties of three stents—the stent of the present invention, the conventional stent, and the Bonastent, and evaluated the migration rates of the stent of the present invention and the Bonastent in an animal model.

All stents were identical in materials and specification; made of nitinol wire of the same diameter (0.203 mm) and silicone membrane; 10 cm in length and 18 mm in diameter with proximal and distal ends of 2 cm in length and 24 mm in diameter. The stent of the present invention is basically the same as the conventional stent. There are 2 to 3 mm-long nonmetallic gaps between the segments of the body, which is connected by 4 metallic rings at intervals of 90°. Each ring is a 1.5 mm×3 mm oval made of nitinol having a diameter of 0.5 mm. This differentiates the stent of the present invention from the conventional stent that has no metallic support. The conventional stent is a conventional stent with 2 interlacing wires from the proximal end to distal end in diagonal direction.

All physical tests were conducted using universal testing machines (Instron Corporation, Norwood, Mass.; Ametek, Lloyd Instruments Ltd., Hampshire, UK; Daekyung Tech & Tester MTG Co., Ltd., Incheon, Korea; Nidec-Shimpo Co., Kyoto, Japan). All tests except the fatigue test were conducted using 3 stents of each type, and the fatigue test was conducted using 2 stents of each.

Statistical analysis was carried out with SAS version 9.13 (SAS Institute, Cary, N.C.) by using the Kruskal-Wallis test and the Tukey test for the results of physical tests, and with Microsoft Excel Software (Microsoft, Redmond, Wash.) by using paired t tests for migration rates.

present invention was approved by the Institutional Animal Care and Use Committee, and all the procedures were conducted in accordance with the eighth edition of the Guide for the Care and Use of Laboratory Animals published by National Research Council of the National Academies, 2011, and followed the guidelines of Samsung Biomedical Research Institute (Seoul, Korea), which has been accredited by the Association for Assessment and Accreditation of Laboratory Animal Care (AAALAC) International.

Longitudinal compressibility was measured by a modified method used in the coronary artery stent. When the stent was compressed by a length of 10 mm longitudinally inside the custom-made jig, the exerted force of the moving part and transferred force to the fixed part on the opposite side were measured. The 3-point bending test was conducted for the measurement of axial force or trans-axial flexibility; trans-axial force needed to displace the middle portion of the stent by 15 mm during fixation of both ends. The lower value of these 2 parameters means better flexibility or compliance of the stent.

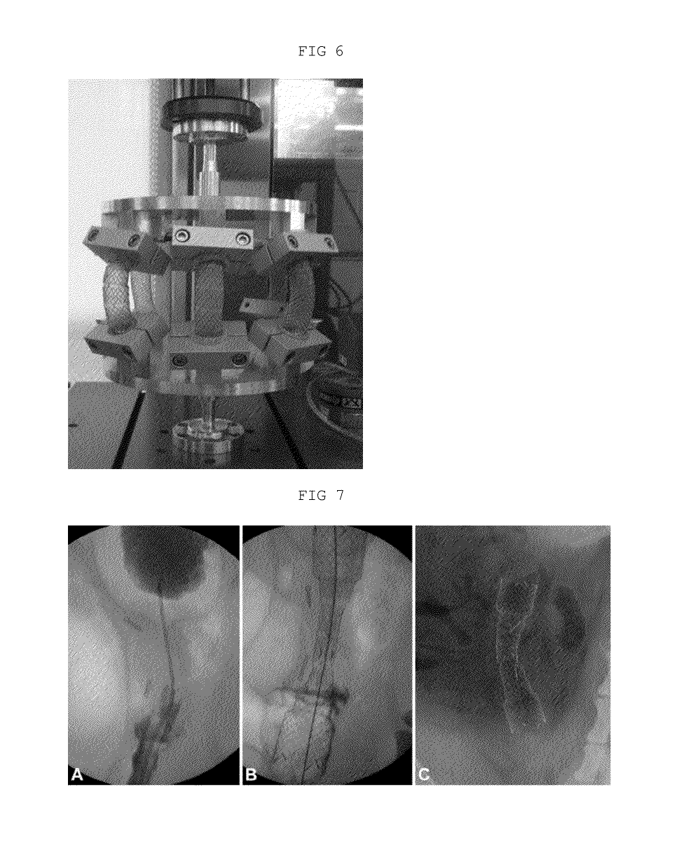

Radial force was measured as the force required to compress the stent by 50% of a diameter by using the flat plate test. The fatigue test was conducted using dynamic force with a linear stroke of 9 mm at an angle of 60° and a frequency of 10 cycles per second for 10 days (FIG. 6). Tensile strength was measured as the minimum force needed to break the stent by pulling each end in opposite directions. Stent shortening was also evaluated by measuring the length of the stent in its loaded and deployed state and the ratio was calculated from the formula: (length of constrained stent minus length of fully deployed stent)/length of constrained stent.

Table 1 is a table showing the results of physical tests. In terms of flexibility, the stent of the present invention exhibited the best results in connection with longitudinal compressibility and axial force. All units are Newton. The number in parenthesis represents the force transferred to the opposite end during compression of the stent by 10 mm of length longitudinally.

TABLE 1

| | Stent of Present | Conventional | |
|---|---|---|---|
| Variable | Invention | Stent | Bonastent |
| Longitudinal | 0.6 (0.5) | 1.8 (1.7) | 6.0 (5.7) |
| compressibility | | | |
| Axial force | 0.85 | 1.66 | 1.95 |
| Radial force | 3.62 | 3.41 | 4.80 |
| Tensile strength | 118.1 | 49 | 635.5 |

* A smaller number is better in terms of longitudinal compressibility and axial force, and a larger number is better in terms of radial force and tensile strength.

Furthermore, the migration rates were compared by installing three stents, including the stent of the present invention, in a colon obstruction part of an animal model and then checking colon obstruction using a contrast agent through fluoroscopy.

The present inventors developed and reported an animal model of GI obstruction effective in evaluating anti-migration properties of stents as well as stent patency. Briefly, a surgical obstruction model was made in healthy mongrel dogs, as follows. The dogs, weighing 19.9 to 28.5 kg (mean 23.3 kg), were acclimated and individually housed for 7 days before experiments. After general anesthesia, a segment of the descending colon was exposed after a lower midline incision. The colon segment was wrapped with a non-absorbable synthetic mesh (Prolene Mesh, Ethicon, Inc., Somerville, N.J.) with the proper length of the mesh according to the diameter of the descending colon of each dog. After the mesh was wrapped around the colon, it was punched to make four holes at each end. The present inventors passed four flat rubber bands through these holes in the mesh and the mesentery. The rubber bands were tightened to induce the complete obliteration of the colon tubule. The present inventors fixed rubber bands with contact adhesives. Surgical suture material was used to put together the mesh and rubber bands and to fix them to the colon wall, and the abdomen was closed surgically. Cefovecin sodium was administered once after surgery and lactulose syrup daily. Four days later, the present inventors confirmed the obstruction of the colon with contrast media under fluoroscopy.

Stent Placement

Dogs were randomly assigned to insertion of the stent of the present inventions in the study group (n=13) and conventional stents (Bonastent) in the control group (n=13). On the fourth day after laparotomy, with the animals under general anesthesia, fluoroscopic examination with contrast media was performed to confirm obstruction of the colon via anal route with a 5F catheter. A 5F angiographic catheter and a 260 cm long, 0.035 inch-diameter hydrophilic guidewire were passed through the obstructed segment. Stents were delivered with a 5 mm-diameter delivery system and placed in the obstructed segments of the colon along the guidewire. After the stent was deployed, a contrast study was obtained to verify the position and patency of the stent. All procedures were done under fluoroscopic guidance by one of the experienced interventions, and the present inventors did not use endoscopy.

Dogs were caged without any restriction of food or activity. Stent patency and migration were evaluated clinically by assessing the dogs and fecal material on a daily basis and fluoroscopically on the seventh day after stent placement. Dogs were euthanized within a few days after stent migration, or on the 14th day, if migration did not occur. The colon was dissected, and the presence of the stent or residual obstruction was confirmed.

Test Results

Physical Properties

The stent of the present invention, in terms of flexibility, showed the best results in both longitudinal compressibility and axial force. In both radial force and tensile strength, the Bonastent ranked in the first place and the stent of the present invention in second place. All differences among these data (Table 1) were statistically significant at P<0.05.

In the fatigue test, fracture occurred in the conventional stent on the third day, and the stent of the present invention and the Bonastent did not break for 10 days. The shortening ratio was 27% in the stent of the present invention and the conventional stent and 29% in the Bonastent.

Outcomes of Animal Study

Stent placement was successful in all dogs without procedural adverse events (FIG. 7). Migration occurred in 2 dogs (15.4%) in the study group and 8 dogs (61.5%) in the control group (P=0.008) after mean day 3.4 (range 1-7 days). All migrated stents were found in the stool. In all dogs without migration, the stents were confirmed in position on fluoroscopy, and the dogs were doing well until euthanized, except 1 dog. In this dog, the stent of the present invention had been inserted, and the next day she developed progressive abdominal distension and finally septic shock. The stent was found in position without any abnormal finding on fluoroscopy. However, closed loop obstruction with the strangulation of the proximal colon was diagnosed at autopsy without any problem in the stent and stented colon, 5 days after stent placement.

The migration of stents has remained the most challenging problem in the placement of covered stents in GI obstruction. Over the past decades, several types of stents focused on anti-migration properties were developed and studied—they are the coaxial double stent, which is composed of an inner covered stent and an outer protuberance of non-covered stent, a stent with an anti-migration collar, and a stent with barbs. Stents of these designs have outer additional parts to fix the stent to the bowel wall, which may cause bowel wall injury or exert excessive pressure to the bowel wall, resulting in adverse events, such as pain, bleeding, or perforation. Unlike these designs focused on the fixation strength of the stent, the novel stent was developed with the idea that both transaxial and longitudinal flexibility is the key to increasing the anti-migration properties of the stent.

the stent of the present invention showed the lowest axial force, which means the highest transaxial flexibility and the tendency to be the most pliable to the bowel wall. That is why the stent of the present invention may adapt well to bowel anatomy, which is one important feature of anti-migration properties. This concept is applied to stent grafts used in endovascular procedures, especially in aortic aneurysm repair. They also have gaps without metallic support between each stent segment, and these gaps are essential elements of stent flexibility, which enable tight contact between stent graft and tortuous vasculature. The novel stent had high longitudinal compressibility. Therefore, the novel stent compresses the most easily by proximal force and transmits it distally the least. This means that the propulsive force or shear stress caused by peristaltic movement of the proximal bowel is considerably absorbed by the stent, and it is less likely to propel the stent distally.

Stent fracture, which is actually the disruption of the silicon covering, is the weakness of the conventional stent. By adding ring connections between stent segments, tensile strength was increased by more than double, and the stent endured the fatigue test for 10 days.

The present inventors examined that in vitro characteristics of the novel stent concerning migration do work in an animal study. The animal model of GI obstruction was made by surgical wrapping of the colon with non-absorbable synthetic mesh, which usually is used in hernia repair. Adequate obstruction was made so that dogs could not defecate well after surgery and could do well after stent placement and survive this experiment. Most of all, the migration rate was much higher than that of human clinical studies.

That means the animal model of the present inventors is a harsh and extreme environment for testing anti-migration properties of stents. Possible explanations can be offered for high migration rates. Although this model simulates malignant bowel obstruction regarding the characteristic findings on fluoroscopic study with contrast media and interventional procedures including passage of a guidewire beyond obstruction, placement, and deployment of a stent, the obstructing mechanism is extrinsic, and there is no mucosal change at all. Thus, friction between the stent and the bowel wall is lower than in intrinsic malignant obstruction. Another explanation is that the dogs were healthy without restriction of diet and activity even when they were caged. Their peristaltic movements might be stronger than those of patients receiving stent placements who were in poor general condition.

In animal experiments, the stent of the present invention showed significantly lower migration rates than the conventional stent, thus reproducing the results of the in vitro test. Although radial force was weak, it was sufficient to keep the tubule patent, and the dogs defecated well.

Stent graft in endovascular aneurysm repair of the aorta has basically a similar structure as the covered stent, and its migration is a graver situation that can cause fatal aortic rupture. Most stent grafts have barbs or hooks in the proximal stent, and they markedly increase the fixation force. However, barbs in GI stents have adverse effects, such as pain, bleeding, perforation, and stenosis caused by granulation tissue formation. Another method of increasing fixation is increasing the radial force of the stent. However, it is limited because it may induce overly tight compression of the bowel wall or reduce the flexibility of the stent, resulting in pressure necrosis or perforation. The concept of the ideal GI stent of the present inventors is one with high flexibility and minimum axial force to keep the tubule patent, which will reduce migration as well as other adverse events without compromising the patency of the stent. In this respect, the stent of the present invention is effective and safe, with a low migration risk.

A limitation of present invention is that it was carried out in a small number of animals with a short follow-up. Concerning the limitation of follow-up time, the previous study (16) of the present inventors demonstrated that the migration of stents occurred in 70.6% (12/17), and 91.7% (11/12) of them occurred within 14 days after stent placement. Therefore, the present inventors thought that this period would be sufficient to compare the anti-migration properties of stents.

The diameter of the delivery system that the present inventors used is 5 mm, and further development is needed for the through-the-scope method.

In conclusion, the stent of the present invention showed better flexibility, both longitudinal and transaxial, in the physical test and lower migration rates in the animal study, than did conventional stents.

Although the specific embodiments of the present invention have been described and illustrated, the present invention is not limited to the described embodiments, but it will be apparent to those having ordinary knowledge in the art that various modifications and variations can be made to the present invention without departing from the spirit and scope of the present invention. Accordingly, these various modifications and variations should be understood as falling within the claims of the present invention.

What is claimed is:

1. A stent, comprising:
a silicon cover formed in an open hollow cylindrical shape in which a diameter of both ends thereof is larger than a diameter of an intermediate portion thereof;
a plurality of cylindrical members mounted on an outer circumference of the silicon cover in a hollow cylindrical shape whose both ends are open, and configured such that a diameter thereof is reduced by external force to be installed in a tubule; and
connection members configured to connect facing ones of the plurality of cylindrical members at regular intervals in a single ring shape between the cylindrical members and to have elasticity in a lengthwise direction.

2. The stent of claim 1, wherein the cylindrical members are each fabricated by disposing one or more lines along a circumferential surface in a zigzag shape to form a plurality of peak parts and a plurality of valley parts.

3. The stent of claim 2, wherein the connection members connect most adjacent peak and valley parts of facing ones of the cylindrical members.

4. The stent of claim 1, wherein the connection members comprise at least three connection members that are connected along circumferences of the cylindrical members.

5. The stent of claim 4, wherein the connection members that connect the cylindrical members are disposed at an same height in a single rectilinear row.

6. The stent of claim 5, wherein the connection members are disposed in two or more rows.

7. The stent of claim 1, wherein the cylindrical members are made of metallic or resin wire that is harmless to a human body.

8. The stent of claim 1, wherein the connection members are made of metallic or resin wire that is harmless to a human body.

9. The stent of claim 7, wherein the metallic wire of the cylindrical members or connection members is made of nitinol or stainless steel.

10. The stent of claim 8, wherein the metallic wire of the cylindrical members or connection members is made of nitinol or stainless steel.

11. The stent of claim 2, wherein the cylindrical members are configured such that when external force is applied, angles of the peak and valley parts are varied, thereby varying a diameter of the cylindrical members.
